(19) United States
(12) Patent Application Publication
(10) Pub. No.: US 2006/0129181 A1
(43) Pub. Date: Jun. 15, 2006

(54) Retrieval device with retractable dilator tip

(76) Inventors: Joseph R. Callol, San Francisco, CA (US); Eliot T. Kim, Santa Clara, CA (US); Richard Renati, Los Gatos, CA (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 11/010,763

(22) Filed: Dec. 13, 2004

(51) Int. Cl. A61M 29/00

(52) U.S. Cl. 606/200

(57) ABSTRACT

Devices and methods for retrieving an intravascular device disposed within a blood vessel are disclosed. A retrieval device in accordance with an illustrative embodiment of the present invention includes an elongated member, a retractable dilator tip, and a retraction wire operatively coupled to the retractable dilator tip. The retraction wire can be connected to a slide mechanism that can be actuated by the physician to expose an interior cavity of the retrieval device that receives the intravascular device therein. In some embodiments, the retrieval device can include an inflatable balloon cuff, a tip deflection wire, and/or other means to facilitate advancement of the retrieval device beyond obstructions within the blood vessel.

### FIELD

The present invention relates generally to the field of medical devices. More specifically, the present invention pertains to devices and methods for retrieving intravascular devices within the body.

### BACKGROUND

Intravascular devices such as embolic protection filters are typically placed in a vessel such as an artery or vein to filter emboli contained in the blood stream. Examples of procedures employing such filters include angioplasty, atherectomy, thrombectomy, and stenting. These procedures generally involve transluminally inserting and delivering a guidewire and embolic protection filter to a location distal a lesion or other stenosis within the vessel. Once placed, a therapeutic device such as an angioplasty catheter can then be advanced along the wire to the site of the lesion to perform a therapeutic procedure such as percutaneous transluminal coronary angioplasty (PTCA). In certain procedures, a stent may also be advanced to the site of the lesion and engaged along the inner wall of the vessel to prevent restenosis from occurring within the vessel.

Retrieval of embolic protection filters generally involves the use of a retrieval device such as a catheter or sheath having an inner lumen configured to collapse the embolic protection filter and any captured emboli therein. The retrieval device is typically tracked over the previously placed guidewire to a location proximal to the deployed embolic protection filter. Once positioned, the embolic protection filter is then retracted into the inner lumen of the retrieval device, causing the embolic protection filter to collapse therein. The retrieval device containing the collapsed embolic protection filter can then be removed from the body.

The ability of the retrieval device to traverse obstructions located within the vasculature can pose a significant challenge for the physician. In interventional procedures employing a stent, for example, the existence of any mal-apposed stent struts along the inner wall of the blood vessel may hinder smooth tracking of the retrieval device along the guidewire, in some cases preventing the retrieval device from being advanced across the stent to the site of the embolic protection filter. Other factors such as the size and shape of the retrieval device may also affect the ability of the device to capture the embolic protection filter.

### SUMMARY

The present invention pertains to devices and methods for retrieving intravascular devices within the body. A retrieval device in accordance with an illustrative embodiment of the present invention includes an elongated member having a proximal section equipped with a handle that can be manipulated by the physician, an intermediate section that includes a side guidewire port, and a distal section defining a distal opening and interior cavity that can be configured to receive an intravascular device such as an embolic protection filter.

A dilator tip movably disposed within the distal section of the elongated member can be used to facilitate tracking of the retrieval device along a guiding member such as a guidewire. The dilator tip can include a proximal section, a distal section, and an inner lumen therethrough. The proximal section of the dilator tip can be dimensioned to provide a positive interference fit with the distal opening of the elongated member, creating a smooth transition between the dilator tip and elongated member that aids in preventing the retrieval device from catching on any obstructions within the blood vessel. The distal section of the dilator tip, in turn, can gradually taper in profile to create a smooth transition between the dilator tip and guidewire.

In certain embodiments, the dilator tip can include an inner guiding member that can be used to thread the guidewire through the side guidewire port formed in intermediate section of the elongated member. The guiding member may have a proximal end movably disposed within a guidewire lumen of the intermediate section, and a distal end disposed within an interior portion of the dilator tip. The guiding member can be formed from a number of layers each exhibiting a desired characteristic. In one illustrative embodiment, for example, the guiding member can include an inner layer of lubricious material to facilitate tracking of the retrieval device along the guidewire, and an outer layer of compatible bonding material that can be used to facilitate bonding of the guiding member to the dilator tip. If desired, one or more intermediate tie layers can also be provided to facilitate bonding of the inner and outer layers.

A retraction wire operatively coupled to the guiding member and/or dilator tip can be provided to actuate the dilator tip between a first position disposed within the distal opening at least in part distally of the elongated member, and a second position located within the interior cavity of the elongated member. A slide mechanism coupled to the retraction wire can be activated by the physician to pull the retraction wire proximally within the elongated member, allowing the dilator tip and intravascular device to be retracted within the interior cavity of the distal section.

The retrieval device can further include one or more features that aid in tracking of the retrieval device beyond obstructions located within the body. In certain embodiments, for example, the distal section of the retrieval device can include an inflatable balloon cuff that can be inflated with pressurized fluid to center the dilator tip within the blood vessel, allowing the physician to traverse the obstruction. In other embodiments, a tip deflection wire operatively coupled to the dilator tip can be used to deflect the positioning of the dilator tip away from the obstruction, similarly allowing the physician to traverse the obstruction.

### BRIEF DESCRIPTION OF THE DRAWINGS

FIG. 1 is a perspective view showing a retrieval device in accordance with an illustrative embodiment of the present invention;

FIG. 2 is a partial longitudinal cross-sectional view showing the retrieval device of FIG. 1;

FIG. 3 is a transverse cross-sectional view showing the retrieval device along line 3-3 in FIG. 1;

FIG. 4 is a transverse cross-sectional view showing the retrieval device along line 4-4 in FIG. 1;

FIG. 5 is a transverse cross-sectional view showing the retrieval device along line 5-5 in FIG. 1;

FIG. 6 is a transverse cross-sectional view showing the retrieval device along line 6-6 in FIG. 1;

FIG. 7 is a longitudinal cross-sectional view showing the dilator tip and guiding member of FIG. 2 in greater detail;

FIG. 8 is a transverse cross-sectional view showing the dilator tip and guiding member along line 8-8 in FIG. 7;

FIG. 9 is a partial longitudinal cross-sectional view showing the retrieval device of FIG. 2 including an inflatable balloon cuff;

FIG. 10 is a partial longitudinal cross-sectional view showing a retrieval device in accordance with another illustrative embodiment of the preset invention including a tip deflection wire;

FIGS. 11-13 are plan views showing an illustrative method of retrieving an intravascular device within a blood vessel using the retrieval device of FIG. 2;

FIGS. 14-16 are plan views showing an illustrative method of traversing an obstruction within a blood vessel using the retrieval device of FIG. 9; and

FIGS. 17-19 are plan views showing an illustrative method of traversing an obstruction within a blood vessel using the retrieval device of FIG. 10.

### DETAILED DESCRIPTION

The following description should be read with reference to the drawings, in which like elements in different drawings are numbered in like fashion. The drawings, which are not necessarily to scale, depict selected embodiments and are not intended to limit the scope of the invention. Although examples of construction, dimensions, and materials are illustrated for the various elements, those skilled in the art will recognize that many of the examples provided have suitable alternatives that may be utilized. While specific devices and methods are described herein for treating particular vascular diseases, it should be understood that the present invention could be used in other treatment modalities requiring the use of intravascular devices.

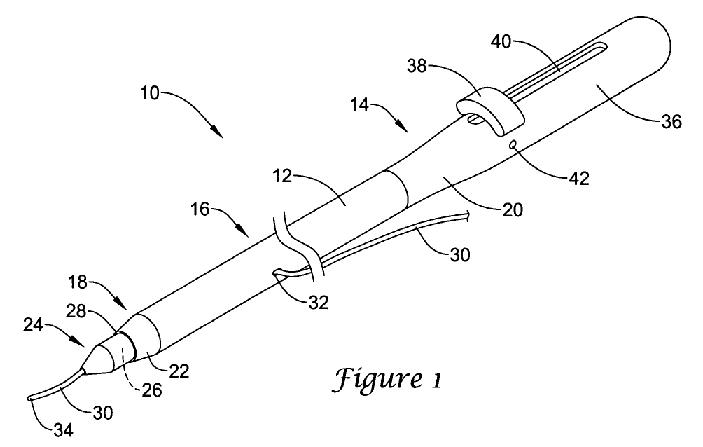

FIG. 1 is a perspective view of a retrieval device 10 in accordance with an illustrative embodiment of the present invention. Retrieval device 10 includes an elongated member 12 having a proximal section 14, an intermediate section 16, and a distal section 18. The retrieval device 10 may have a substantially circular profile that tapers distally at one or more locations along its length to transition the retrieval device 10 from a relatively large profile along the proximal section 14 of the elongated member 12 to a relatively small profile along the distal section 18 thereof. In the illustrative embodiment depicted in FIG. 1, for example, a first tapering section 20 of the retrieval device 10 may reduce the profile of the elongated member 12 along a portion of the proximal section 14. A second tapering section 22 of the retrieval device 10 can be provided to reduce the profile of the elongated member 12 along a portion of the distal section 18 thereof.

While two tapering sections 20,22 are depicted in the illustrative embodiment of FIG. 1, it should be understood that a greater or lesser number of tapering sections, or alternatively a continuous taper, can be provided along the length of the elongated member 12. By tapering the profile of the retrieval device 10 either continuously or along one or more discrete sections of the elongated member 12, a greater amount of stiffness and torsional rigidity can be provided along the proximal section. In use, this increased stiffness and torsional rigidity facilitates the efficient transference of axial and rotational movement through the proximal section 14 as the physician manipulates the retrieval device 10 within the body.

The retrieval device 10 may further include a dilator tip 24 which, as described in greater detail below, can be used to facilitate tracking of the retrieval device 10 through the vasculature, and which can be used to aid in retrieving an intravascular device (e.g. an embolic protection filter) disposed within a blood vessel. As shown in a first (i.e. deployed) position for use in tracking the retrieval device 10 through the patient's vasculature, the dilator tip 24 may extend at least in part distally from a distal opening 26 formed in the distal end 28 of the elongated member 12, and may define an interior lumen adapted to slidably receive a guidewire 30 or other suitable guiding member. In use, the dilator tip 24 provides a gradual transition in profile that prevents interference from occurring as the retrieval device 10 is tracked along the guidewire 30 beyond other intravascular devices. The dilator tip 24 further aids in maintaining the retrieval device 10 in a centered position along the guidewire 30, further improving tracking of the device 10 through the vasculature.

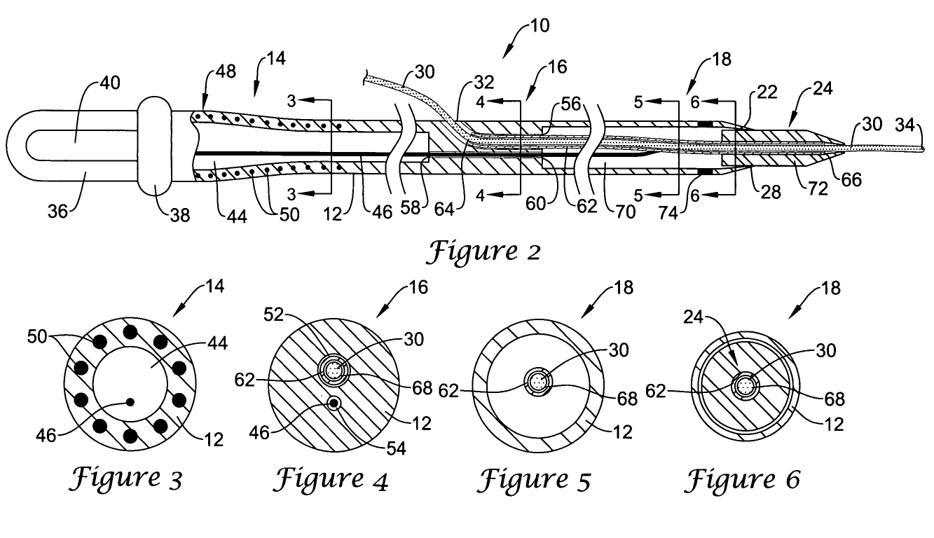

The retrieval device 10 can be configured to permit advancement through the body using either a fixed wire approach or a single operator exchange approach. In the illustrative embodiment of FIG. 1, for example, a skived guidewire port 32 disposed in the sidewall of the intermediate section 16 can be provided to permit the retrieval device 10 to be advanced via a single operator exchange approach along the guidewire 30, allowing the physician to easily grasp the guidewire 30 while manipulating the retrieval device 10 through the vasculature. As is described in greater detail with respect to FIG. 2, the retrieval device 10 can be configured to permit the distal end 34 of guidewire 30 to be inserted through the guidewire port 32 and advanced through the dilator tip 24, or in the alternative, by backloading the proximal end of the guidewire 30 proximally through the dilator tip 24 and out the guidewire port 32. The guidewire port 32 can be skived in a manner that permits the portion of the guidewire 30 located proximally of the guidewire port 32 to lie substantially adjacent and parallel to wall of the elongated member 12 so that the retrieval device 10 maintains a low profile within the body.

The proximal section 14 of the retrieval device 10 can include a handle 36 that can be used by the physician to manipulate the retrieval device 10 from a position outside of the patient's body. A thumbpiece 38 slidably disposed within a slot 40 formed in the handle 36 can be provided as part of a slide mechanism that can be used actuate the dilator tip 24 between the deployed position depicted in FIG. 1 and a second (i.e. retracted) position within the distal section 18 of the elongated member 12. The slide mechanism can be configured such that when the thumbpiece 38 is positioned furthest forward or distally within the slot 40, the dilator tip 24 is extended the furthest distally from the distal end 28 of the elongated member 12. In certain embodiments, the retrieval device 10 may include an internal spring mechanism that permits only movement of the thumbpiece 38 in the proximal direction within the slot 40. A reset pin 42 or other means for resetting the internal spring mechanism can be provided to reset the location of the thumbpiece 38 within the slot 40, if desired, allowing the physician to reposition the thumbpiece 38 back to its default position depicted generally in FIG. 1.

FIG. 2 is a partial longitudinal cross-sectional view showing the retrieval device 10 of FIG. 1 in greater detail. As can be seen in FIG. 2, the proximal section 14 of the elongated member 12 may define an interior lumen 44 which houses the slide mechanism as well as a retraction wire 46 that can be used to retract the dilator tip 24 back and forth within the distal section 18 of the elongated member 12. As can be seen in FIG. 3, the interior lumen 44 of the proximal section 14 may be sized sufficiently to permit the retraction wire 46 to move in response to movement of the thumbpiece 38 back and forth within the slot 40.

The proximal section 14 may be formed from a suitable stiff material having sufficient column strength and rigidity to withstand buckling or bulging as the retrieval device 10 is advanced over the guidewire 30 and engaged against an intravascular device. The proximal section 14 may be formed at least in part from a polymeric material such as polyether block amide (PEBA), which is commercially available from Atochem Polymers of Birdsboro, Pa. under the trade name PEBAX. Other suitable polymeric and/or metallic materials frequently used in the construction of catheters shafts or retrieval sheaths may also be employed. Examples of other suitable materials may include, but are not limited to, polyethylene terapthalate (PET), polytetrafluoroethylene (PTFE), polyurethane (Nylon), fluorinated ethylene propylene (FEP), ethylene tetrafluoroethylene (ETFE), polyurethane, polypropylene (PP), polyvinylchloride (PVC), polyether-ester, polyester, polyamide, elastomeric polyamides, block polyamide/ethers, polyether block amide (PEBA), silicones, polyethylene (PE), polyether-ether ketone (PEEK), polyimide (PI), polyetherimide (PEI), polyphenylene sulfide (PPS), polyphenylene oxide (PPO), polysulfone, perfluoro(propyl vinyl ether) (PFA), or other suitable materials, mixtures, combinations or copolymers thereof. In certain embodiments, the polymeric material may be blended with or otherwise include a liquid crystal polymer (LCP) to enhance torqueability.

The material used to form the proximal section 14 may differ from the material used to form the intermediate and/or distal sections 16,18 to impart a particular characteristic to the retrieval device 10. In certain embodiments, for example, the material forming the proximal section 14 may have a relatively low modulus of rigidity and elasticity than the material forming the intermediate and/or distal sections 16,18, imparting greater stiffness and torqueability to the proximal section 14. By varying the selection of materials in this manner, the intermediate and/or distal sections 16,18 comprising the relatively flexible material can be configured to undergo a greater amount of bending or flexion to facilitate tracking of the retrieval device 10 into difficult to reach areas of the vasculature such as a branching vessel.

A strain relief member can be provided along all or portions of the proximal section 14 to further increase the rigidity of the elongated member 12 along this section. In the illustrative embodiment of FIG. 2, for example, a braided member 48 including a number of filaments 50 encased within the wall of the proximal section 14 can be used to provide additional rigidity to the elongated member 12 near the handle portion of the retrieval device 10. While the braided member 48 depicted in FIG. 2 is shown terminating at a location proximal to the intermediate section 16, other embodiments have been contemplated where the braided member 48 extends along all or portions of the intermediate and/or distal sections 16,18 of the elongated member 12.

The filaments 50 can be made from any number of suitable materials including polymers, metals, metal alloys, metal-polymer composites, or metal-metal composites. Some examples of suitable metals and metal alloys include platinum, stainless steel, nickel-titanium alloy (Nitinol), nickel-chromium alloy, nickel-chromium alloy, cobalt alloy, or the like. Polymers such as that used in the construction of the elongated member 12 may also be used in forming the filaments 50.

Other characteristics such as the shape and thickness of the filaments 50 may also be varied to alter the characteristics of the retrieval device 10. In the illustrative embodiment depicted in FIG. 2, the filaments 50 forming the braided member 48 are made from monofilament wire having a generally round transverse cross-sectional area. Other filament configurations may be employed, however, such as flat ribbon, multi-filament wire, threads, fibers, or combinations thereof.

The intermediate section 16 of the elongated member 12 may define a first lumen 52 adapted to slidably receive the guidewire 30, and a second lumen 54 adapted to slidably receive the retraction wire 46. The first lumen 52 may extend distally from the guidewire port 32 formed through the sidewall of the elongated member 12 to a distal end 56 located at or near the interface between the intermediate section 16 and the distal section 18. The second lumen 54, in turn, may extend distally from a proximal end 58 to a distal end 60 thereof.

In certain embodiments, a guiding member 62 disposed at least in part within the first inner lumen 52 can be used to facilitate tracking of the guidewire 30 through the retrieval device 10 and dilator tip 24. The guiding member 62 can include a proximal end 64 disposed within the first lumen 52 of the intermediate section 16, and a distal end 66 disposed within the interior of the dilator tip 24. As can be seen in greater detail in FIG. 4, the inner diameter of the first lumen 52 can be slightly larger than the outer diameter of the guiding member 62, allowing the guiding member 62 to move back and forth within the first lumen 52 during retraction of the dilator tip 24. An inner lumen 68 disposed within the guiding member 62, in turn, can be configured to slidably receive the guidewire 30.

The distal section 18 of the elongated member 12 may define an interior cavity 70 adapted to receive and contain an intravascular device therein. The dimensions of the interior cavity 70 may vary depending on the size of the intravascular device to be captured as well as the dimensions of the dilator tip 24. As can be further seen in FIG. 2 and in FIGS. 5-6, the thickness of the wall along the distal section 18 of the elongated member 12 can be feathered such that the wall thickness of the elongated member 12 is reduced from the onset of the second tapering region 22 to the distal end 28 thereof. In certain embodiments, such configuration can be used to taper the inner diameter of the elongated member 12 at or near the distal opening 26 to a size slightly smaller than a proximal section 72 of the dilator tip 24, providing a positive interference fit therebetween. This positive interference fit along the thinned-down portion of the distal section 18 creates a smooth transition between the dilator tip 24 and the elongated member 12 that aids in preventing the retrieval device 10 from catching on any obstructions as it is advanced through the vasculature. In addition, such interference fit serves to prevent any saline injected within the interior cavity 70 from leaking out through the guidewire port 32 and/or through the distal opening 26 as the retrieval device 10 is advanced through the vasculature.

The material or combination of materials forming the distal section 18 of the retrieval device 10 can also be selected to form a positive interference fit between the distal opening 26 of the elongated member 12 and the dilator tip 24. In certain embodiments, for example, an elastomeric polymer such as polyether block amide (PEBAX) can be employed, allowing the distal section 18 to be radially stretched and enlarged during sheath fabrication so that the dilator tip 24 can be inserted within the elongated member 12, and then relaxed to create a positive interference fit with the proximal section 72 of the dilator tip 24.

To permit visualization within the body, at least a portion of the distal section 18 can be loaded with or otherwise formed of a radiopaque material. Examples of suitable radiopaque materials can include tungsten, bismuth subcarbonate ((BiO)₂CO₃) and barium sulfate (BaSO₄). The amount of loading may vary between 25% to 90% by weight depending on the amount of loading necessary to generate sufficient visibility under normal fluoroscopic conditions. Other factors such as the length of the dilator tip 24 and the wall thickness of the elongated member 12 at the distal section 18 will also affect the amount of loading.

In certain embodiments, the retrieval device 10 may include a radiopaque marker band 74 formed at a location proximal to the distal end 28 of the elongated member 12. The marker band 74 can include a radiopaque material (e.g. platinum, gold, tantalum, tungsten, etc.) that can be used to fluoroscopically judge the location of the retrieval device 10 within the vasculature. The marker band 74 can be positioned sufficiently close to the distal end 28 of the elongated member 12 so that the physician can approximate the location of the dilator tip 24 as the retrieval device 10 is advanced through the body. In certain embodiments, for example, the radiopaque marker band 74 can be positioned at a distance of about 0.5 mm to 10 mm, and more specifically about 2-3 mm from the distal end 28 of the elongated member 12, although other placement positions greater or lesser than this distance are possible. In use, the marker band 74 can be used by the physician to gauge the distance that the retrieval device 10 is inserted into the vasculature during delivery, preventing the physician from over-advancing the retrieval device 10 against the intravascular device. In addition, the marker band 74 can be used by the physician to approximate the distance that the intravascular device is withdrawn within the interior cavity 70 during retrieval. In some embodiments, a radiopaque marker band (not shown) on the guidewire 30 can be configured to align with the marker band 74 on the distal section 18, providing the physician with feedback that the intravascular device has been retracted into the interior cavity 70. While only one marker band 74 is depicted in the illustrative embodiment of FIG. 2, it should be understood that multiple radiopaque marker bands could be employed at one or more locations along the length of the elongated member 12, if desired.

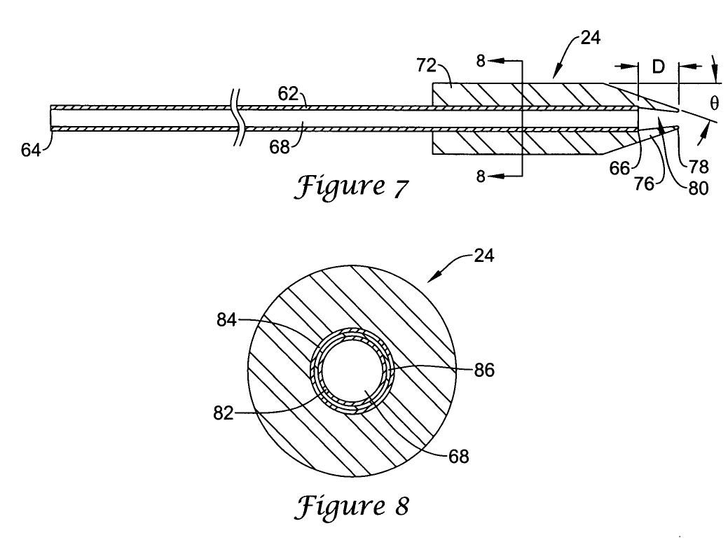

FIG. 7 is a longitudinal cross-sectional view showing the dilator tip 24 and guiding member 66 of FIG. 2 in greater detail. As can be seen in FIG. 7, the dilator tip 24 can include a tapering distal section 76 that gradually transitions the profile of the dilator tip 24 from a relatively large, constant diameter profile along the proximal section 72 to a relatively small profile at or near the distal end 78 thereof. In use, such transition in profile along the tapered distal section 76 reduces trauma to the body as the retrieval device 10 is advanced through the vasculature, and further aids in manipulating the retrieval device 10 through tortuous anatomy, small vessels, calcified lesions, mal-apposed stent struts, etc.

As can be further seen in FIG. 7, the guiding member 62 may extend through a majority of the length of the dilator tip 24, terminating at a location proximal to the distal end 78 thereof. The distal end 66 of the guiding member 62 can be spaced apart from the distal end 78 of the dilator tip 24 by a distance D, which is typically a few millimeters in length. The distance D at which the distal end 66 of the guiding member 62 terminates proximally of the distal end 78 of the dilator tip 24 may depend in part on the angle θ at which the tapering distal section 76 slopes away from the proximal section 72. In general, the steeper the angle θ at which the tapering distal section 76 slopes away from the proximal section 72, the smaller the distance D at which the guiding member 62 terminates proximally from the distal end 78 of the dilator tip 24, although other configurations are possible. Other factors such as the overall length of the dilator tip 24 will also affect the distance D at which the guiding member 62 terminates proximally from the distal end 78 of the dilator tip 24.

The guiding member 62 can be dimensioned to permit the retrieval device 10 to be advanced along a variety of sized guidewires commonly used in the art. In certain embodiments, for example, the inner lumen 68 of the guiding member 62 may have a diameter in the range of about 0.015 inches to 0.017 inches, which is sufficient to accept guidewires (i.e. filter wires) commonly used in conjunction with embolic protection filters. It should be understood, however, that the dimensions of the inner lumen 68 can be made greater or smaller to accommodate other sized guidewires, or to permit the use other types of guiding members.

An inner portion 80 of the dilator tip 24 lumen extending distally from the distal end 66 terminus of the guiding member 62 to the distal end 78 of the dilator tip 24 may gradually taper such that the inner diameter of the dilator tip 24 at the inner portion 80 is slightly smaller than the inner diameter of the guiding member 62. In certain embodiments, for example, the lumen exit at the distal end 78 of the dilator tip 24 may have an inner diameter of about 0.013 inches to 0.015 inches whereas the guiding member 62 may have an inner diameter of about 015 inches to 0.017. It should be understood, however, that the dimensions of the inner lumen 68 of the guiding member and inner portion 80 of the dilator tip 24 may vary based on the size of the guidewire as well as other factors. In use, the reduction in size at the inner portion 80 serves to reduce the annulus between the guidewire and the distal end 78 of the dilator tip 24.

The dilator tip 24 can be manufactured using a molding or insert molding process, or by an in-situ molding or shaping process utilizing induction, hot air, or lower energy to mold and shape the materials. Other manufacturing processes such as heat bonding, heat forming, tube necking, swaging, adhesive bonding, ultrasonic welding, RF welding, dip coating, and/or laser forming can also be employed, if desired. In one illustrative molding process, for example, a core pin coated with a number of desired layers of material can be used to form a transition in stiffness along the length of the dilator tip 24. In some cases, such process could also be used to apply a layer or coating of lubricious material to the contact surfaces of the guiding member 62 and dilator tip 24. In an illustrative in-situ process, fabrication of the dilator tip 24 can be accomplished by laminating the polymer layers together over a core pin using a single or multi-step process. A heat shrink tube can be provided over the material layers to provide the necessary pressure and shape during lamination. In those embodiments where the dilator tip 24 includes a taper, multiple heat shrink tubes can be used.

The dilator tip 24 can be fabricated from a suitable polymer or polymer composition including, but not limited to, PEBAX, nylon, polyurethane, polyethylene, etc. The dilator tip 24 can have a uniform composition along its length, or can vary in a manner similar to that described above with the elongated member 12. In the latter case, for example, the tapering distal section 76 of the dilator tip 24 can be made more flexible than the proximal section 72 to facilitate advancement of the retrieval device 10 through the body.

To permit visualization within the body using a fluoroscope, all or a portion of the dilator tip 24 can be loaded with a radiopaque material. In certain embodiments, for example, the dilator tip 24 may be loaded with tungsten, bismuth subcarbonate ((BiO)₂CO₃), barium sulfate (BaSO₄) or other suitable radiopaque agent. In use, such radiopacity can be used to visualize the location of the dilator tip 24 within the body, informing the physician when the retrieval device 10 has reached the correct position within the body for retracting the dilator tip 24 and retrieving the intravascular device. In addition, such radiopacity of the dilator tip 24 may serve as an aid to negotiate obstacles within the body such as mal-apposed stents that may prevent the retrieval device 10 from being advanced, allowing the physician to take appropriate corrective action, if necessary.

FIG. 8 is a transverse cross-sectional view showing the dilator tip 24 and guiding member 62 along line 8-8 in FIG. 7. As can be seen in FIG. 8, the guiding member 62 may have a multi-layer construction including an inner layer 82 of lubricious material having a low coefficient of friction (e.g. PTFE or PE) to reduce friction between the contact surfaces of the guidewire and the guiding member 62, and an outer layer 84 of polymeric material having good bonding compatibility with the material forming the dilator tip 24. In certain embodiments, an intermediate layer 86 can be provided between the inner and outer layers 82,84. Such intermediate layer 86 can be provided, for example, as a tie layer in those instances where the inner and outer layers 82,84 are formed of incompatible bonding materials. In one illustrative embodiment, for example, an intermediate layer 86 of polyolefin such as PLEXAR® or PRIMACORE™ can be employed as the intermediate layer 86 to tie an inner layer 82 and outer layer 84 comprising of polyethylene (PE) and polyether block amide (PEBA), respectively. The selection of material(s) forming the intermediate layer 86 can be varied depending on the types of materials used for the inner and outer layers 82,84, and the type of manufacturing process employed.

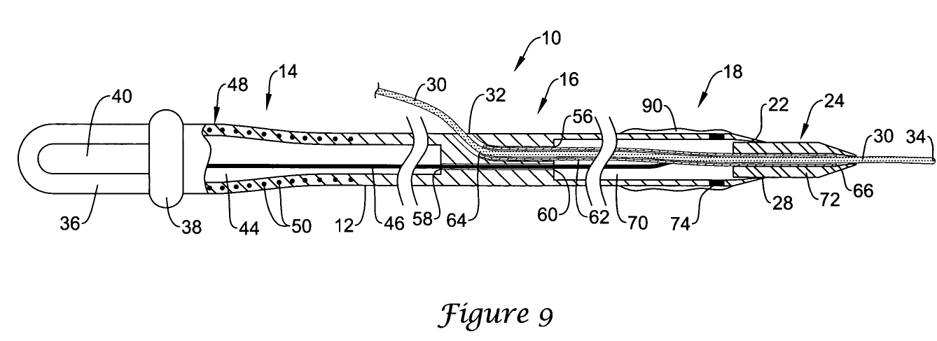

FIG. 9 is a partial longitudinal cross-sectional view showing the retrieval device 10 of FIG. 2 including an inflatable balloon cuff 90 that can be used to aid in tracking the retrieval device 10 beyond obstructions located within the vasculature. As can be seen in FIG. 9, the balloon cuff 90 can be positioned about the outer surface of the elongated member 12 at a location proximal to the distal end 28 thereof. The balloon cuff 90 can be fluidly connected to an inflation lumen (not shown) disposed within the interior of the elongated member 12 that can be used to deliver pressurized fluid (e.g. saline solution) to the balloon cuff 90 at selective times as the retrieval device 10 is advanced through the vasculature. An inflation/deflation source such as a syringe or indeflator can be provided to selectively inflate or deflate the inflatable balloon 90, as desired.

The balloon cuff 90 can be made from an elastomeric material or compliant material that can be expanded when pressurized with inflation fluid. The overall profile of the balloon cuff 90 can be made sufficient to permit the physician to traverse an obstruction within the vasculature such as a mal-apposed stent while still permitting the retrieval device 10 to be advanced across the site of the obstruction. In certain embodiments, for example, the balloon cuff 90 can have an expanded diameter that is generally less than 2.0 mm, allowing the retrieval device 10 to be tracked along the guidewire 30 while the balloon cuff 90 is still inflated. When deflated, the balloon cuff 90 can assume a relatively small profile against the outer surface of the elongated member 12.

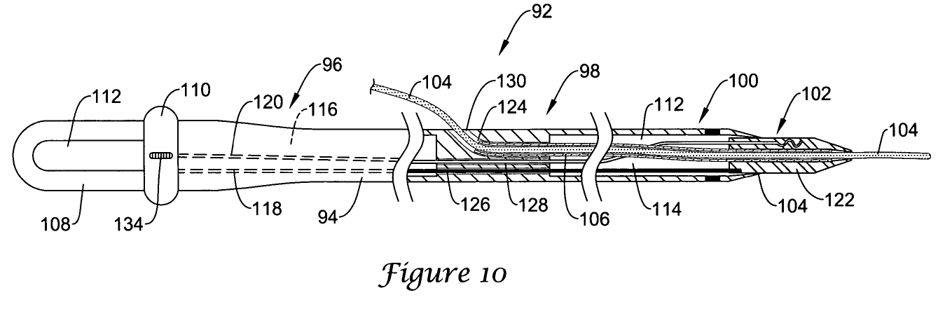

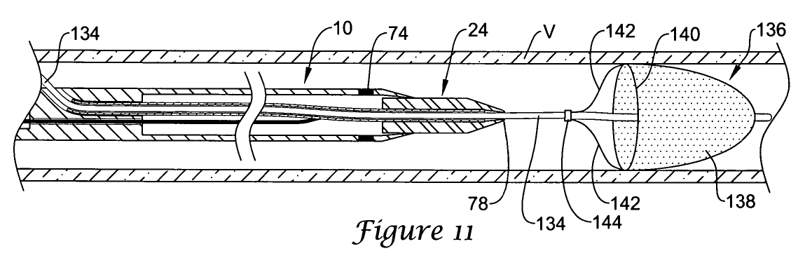

FIG. 10 is a partial longitudinal cross-sectional view showing a retrieval device 92 in accordance with another illustrative embodiment of the present invention including a tip deflection wire. Retrieval device 92 can be configured similar to retrieval device 10 described above, including an elongated member 94 having a proximal section 96, an intermediate section 98, and a distal section 100. As with other embodiments herein, the retrieval device 92 can include a dilator tip 102 that can be used to facilitate tracking of the retrieval device 92 along a guidewire 104, and that can be used to aid in retrieving an intravascular device such as an embolic protection filter disposed within the body. The dilator tip 102 can be configured to extend at least in part distally from a distal end 104 of the elongated member 94, and may include a guiding member 106 adapted to slidably receive the guidewire 104.

The proximal section 96 of the elongated member 94 can include a handle 108 that can be used by the physician to manipulate the retrieval device 92 from a location outside of the patient's body. A thumbpiece 110 slidably disposed within a slot 112 formed in the handle 108 can be used to actuate the dilator tip 102 between a deployed position and a retracted position to expose an interior cavity 114 of the distal section 100. As with retrieval device 10, retrieval device 92 may also include an internal spring mechanism that permits only movement of the thumbpiece 110 in the proximal direction within the slot 112. A reset pin or other means for resetting the internal spring mechanism back to its default position illustrated in FIG. 10 can be further provided, if desired.

The proximal section 96 of the elongated member 94 may define an interior lumen 116 that houses the thumbpiece 110, a retraction wire 118, a tip deflection wire 120, as well as other components of the slide mechanism. In the illustrative embodiment of FIG. 10, the retraction wire 118 is connected at a proximal end to the thumbpiece 110, and at a distal end to a proximal section 122 of the dilator tip 102. In an alternative embodiment, the retraction wire 118 can be connected to the guiding member 106 in a manner similar to that depicted, for example, in FIG. 2.

The intermediate section 98 of the elongated member 12 may define a first lumen 124 adapted to slidably receive the guiding member 106 and guidewire 104, a second lumen 126 adapted to slidably receive the retraction wire 118, and a third lumen 128 adapted to slidably receive the tip deflection wire 120. A skived guidewire port 130 formed in the sidewall of the intermediate section 98 can be provided to permit the retrieval device 92 to be advanced along the guidewire 104 via a single operator exchange approach, allowing the physician to easily grasp the guidewire 104 while manipulating the retrieval device 92 through the vasculature. It should be understood, however, that the retrieval device 92 could be configured to permit tracking along the elongated guidewire 104 using a fixed wire approach, if desired.

The tip deflection wire 120 can be configured to permit the physician to deflect the dilator tip 102 within the distal opening at the distal end 104 of the elongated member 94, allowing the physician to steer around stent struts or other obstacles located within the vasculature. The tip deflection wire 120 can be coupled at a proximal end to a thumbwheel 132 located on the thumbpiece 110, which can be engaged by the physician's finger to either retract or advance the tip deflection wire 120 within the elongated member 94. In the illustrative embodiment of FIG. 10, the distal end of the tip deflection wire 120 can have a serpentine-like pattern to facilitate bonding of the tip deflection wire 120 to the dilator tip 102.

In use, the tip deflection wire 120 can be manipulated by the physician to apply tension to one side of the dilator tip 102, causing the dilator tip 102 to move within the distal opening of the elongated member 94. Such tip deflection can be used, for example, to manipulate the retrieval device 10 beyond a mal-apposed stent strut or other obstruction within the body. The thumbwheel 124 can be mounted to the thumbpiece 110 in a manner that permits the physician to simultaneously retract the retraction wire 118 and tip deflection wire 120 while the thumbpiece 110 is moved proximally within the slot 112.

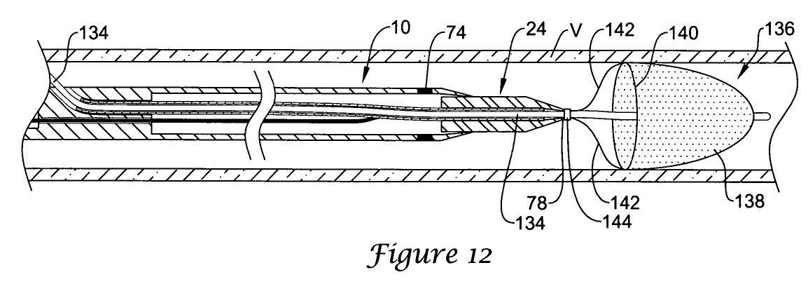

Referring now to FIGS. 11-13, an illustrative method of retrieving an intravascular device will now be described with respect to the illustrative retrieval device 10 described hereinabove. In a first position depicted in FIG. 11, the retrieval device 10 is shown advanced along a filter wire 134 to a location within a blood vessel V proximal to a deployed embolic protection filter 136. Embolic protection filter 136 may include a filter membrane 138 operatively coupled to a support hoop 140 that supports the filter membrane 138 in an expanded position within the blood vessel V. The support hoop 140 can be configured to self-expand when unconstrained radially, biasing the filter membrane 138 to expand outwardly within the vessel V. The support hoop 140, in turn, may be connected to the filter wire 134 via one or more struts 142 extending proximally from the support hoop 140 to a stop 144. Stop 144 can include a clamp or wire winding, solder or other suitable connector coupling the proximal portion of the embolic protection filter 136 to the filter wire 134. The portion of the filter membrane 138 located at or near the distal end of the embolic protection filter 136, in turn, can be attached to the filter wire 134 by adhesion or other suitable bonding technique.

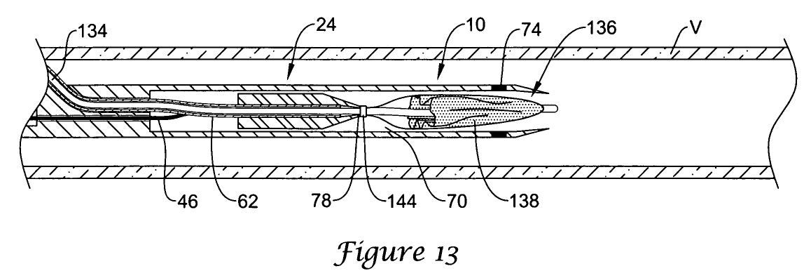

To retrieve the embolic protection filter 136 from the blood vessel V, the physician, while holding the filter wire 136 stationary, may advance the retrieval device 10 distally along the filter wire 134 until the distal end 78 of the dilator tip 24 engages or is near the stop 144 on the filter wire 134, as shown, for example, in FIG. 13. If desired, a fluoroscopic monitor can be used to visualize the location of the dilator tip 24 within the vessel, preventing the physician from over-inserting the retrieval device 10 beyond the location of the embolic protection filter 136. In some embodiments, for example, the physician may visualize the precise location of the retrieval device 10 within the vasculature using the radiopaque marker band 74 on the elongated member 12 and/or the dilator tip 24.

Once the dilator tip 24 has been engaged against the stop 144 of the filter wire 134, the physician, while holding the retrieval device 10 stationary, may retract the thumbpiece 38 proximally within the slot 40 (see FIG. 2), causing the retraction wire 46 to pull the guiding member 62 and attached dilator tip 24 proximally within the interior cavity 70, thereby exposing the interior cavity 70 to the embolic protection filter 136. Continued retraction of the thumbpiece 38 in this manner causes the filter membrane 138 and other components of the embolic protection filter 136 to collapse at least in part within the interior cavity 70, as shown, for example, in FIG. 13. Once disposed within the interior cavity 70, the physician can then remove the retrieval device 10 and embolic protection filter 136 from the body.

In certain embodiments, the dilator tip 24 can be configured to act as a hard stop during retraction of the embolic protection filter 136 into the interior cavity 70. As shown in FIG. 13, for example, the intermediate section 16 of the elongated member 12 can be configured to prevent further proximal movement of the dilator tip 24 and/or embolic protection filter 136 within the interior cavity 70, thus ensuring that excessive forces are not imposed on the embolic protection filter 136 in the event of a full or overly-full filter membrane 138. Such hard stop can also be used to provide the physician with tactile feedback that the embolic protection filter 136 has been retrieved within the retrieval device 10.

While FIGS. 11-13 illustrate the retrieval of an embolic protection filter 136 from the body, it is contemplated that any number of other intravascular devices may be retrieved and/or delivered using the retrieval device 10. Examples of other intravascular devices that can be retrieved include, but are not limited to, stents, clot pullers, vena cava filters, atherectomy devices, angioplasty devices, or the like. While retrieval device 10 is specifically depicted in the various steps, it should be understood, however, that any of the retrieval devices described herein (or variations thereof) can be used to retrieve the embolic protection filter 136.

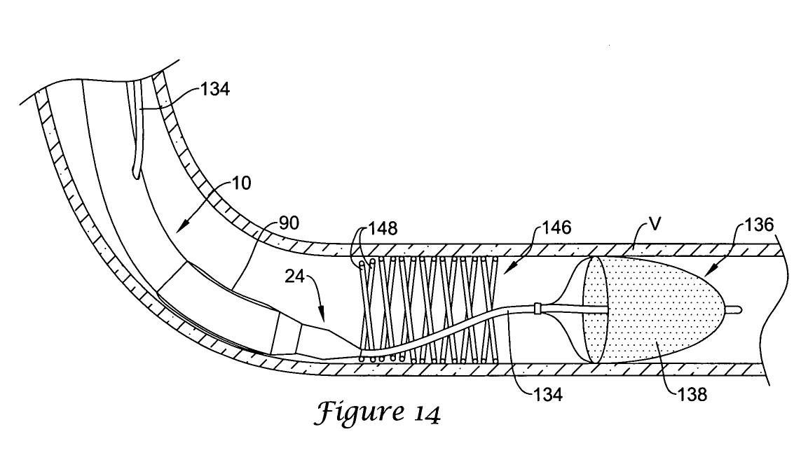

FIGS. 14-16 are plan views showing an illustrative method of traversing an obstruction within a blood vessel V using the retrieval device 10 of FIG. 9. In a first position depicted in FIG. 14, the retrieval device 10 is show advanced along the filter wire 134 to a position proximal a mal-apposed stent 146 implanted within the blood vessel V at a location distal a bend or curve. The stent 146 may include a number of stent struts 148, some of which may become mal-apposed along the inner wall of the blood vessel V. As the retrieval device 10 tracks over the filter wire 134 in the vicinity of the stent 146, interference may result between the retrieval device 10 and the stent struts 148. In some cases, such interference may hinder the ability of the retrieval device 10 to cross the stent 146 and retrieve the embolic protection filter 136.

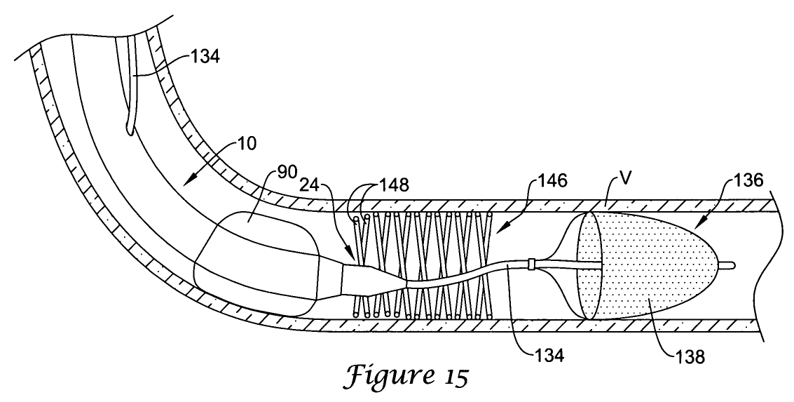

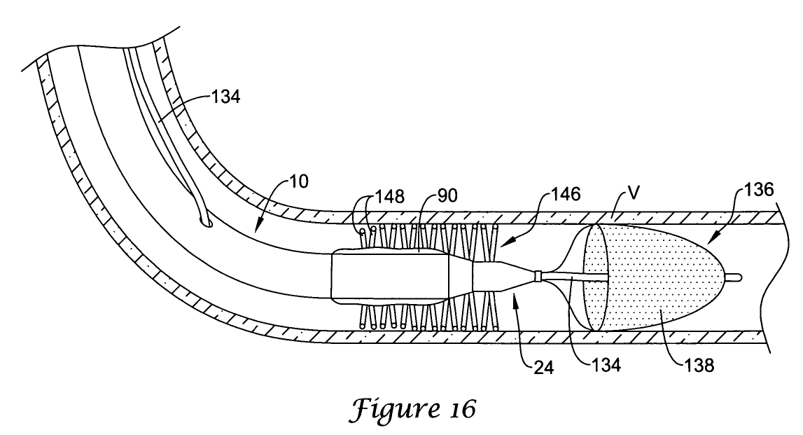

To traverse the obstructive stent 146, the physician may inflate the balloon cuff 90 to a second (i.e. expanded) position, causing the dilator tip 24 to lift off of the inner wall of the blood vessel V, as shown, for example, in FIG. 15. Once the dilator tip 24 has cleared the mal-apposed stent struts 148, the physician may then deflate the balloon cuff 90 and continue to advance the retrieval device 10 distally across the site of the stent 146, as shown in a subsequent position depicted, for example, in FIG. 16. The physician can then retrieve the embolic protection filter 136 in a manner similar to that described above with respect to FIGS. 11-13.

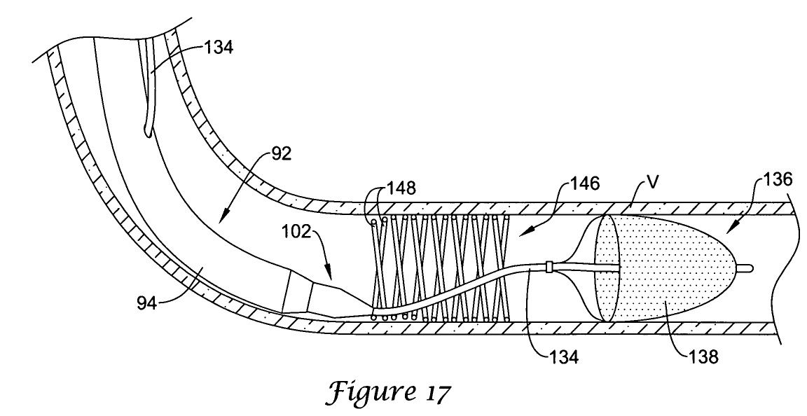

FIGS. 17-19 are partial longitudinal cross-sectional views showing an illustrative method of traversing an obstruction within a blood vessel V using the retrieval device 92 described above with respect to FIG. 10. In a first position illustrated in FIG. 17, the retrieval device 92 is shown advanced along the filter wire 134 to a position proximal a mal-apposed stent 146 implanted within the blood vessel V, similar to that described above, for example, in FIG. 14.

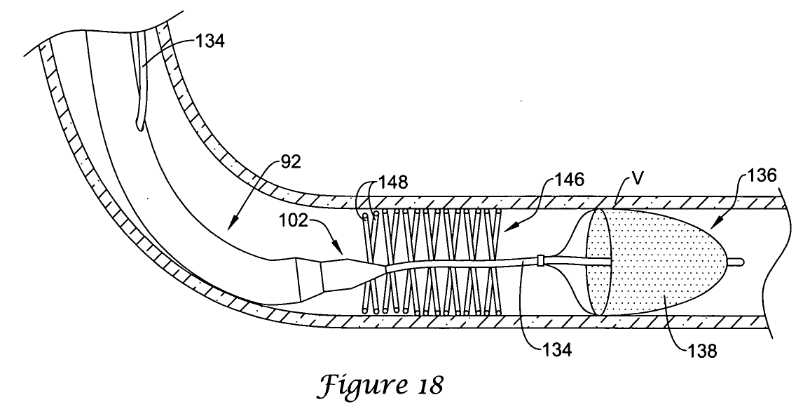

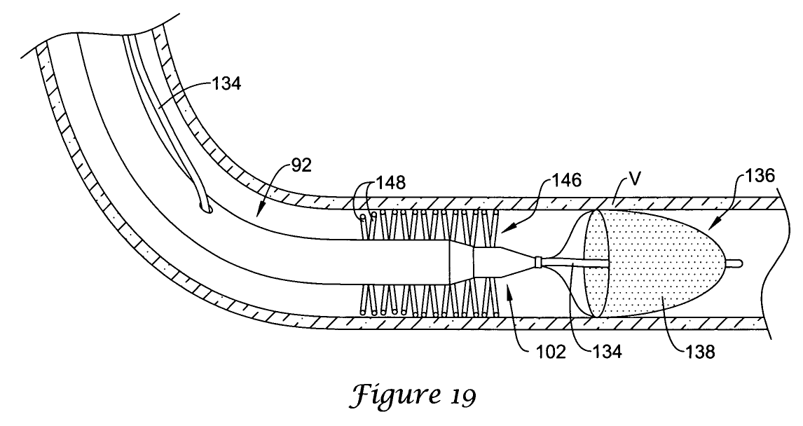

To traverse the obstructive stent 146, the physician, while holding the elongated member 94 and filter wire 134 stationary, retracts the tip deflection wire 120 (see FIG. 10) proximally, causing the dilator tip 102 to deflect away from the inner wall of the blood vessel V, as shown, for example, in FIG. 18. Retraction of the tip deflection wire 110 can be accomplished by rotating the thumbwheel 134 while holding the thubmpiece 110 stationary. Once the dilator tip 102 has been deflected, the physician can then continue to advance the retrieval device 92 distally across the site of the stent 146, as shown in a subsequent position in FIG. 19. The physician can then retrieve the embolic protection filter 136 in a manner similar to that described above with respect to FIGS. 11-13.

Having thus described the several embodiments of the present invention, those of skill in the art will readily appreciate that other embodiments may be made and used which fall within the scope of the claims attached hereto. Numerous advantages of the invention covered by this document have been set forth in the foregoing description. It will be understood that this disclosure is, in many respects, only illustrative. Changes may be made in details, particularly in matters of shape, size and arrangement of parts without exceeding the scope of the invention.

What is claimed is:

1. A retrieval device, comprising:
an elongated member having a proximal section, an intermediate section, and a distal section, said distal section defining a distal opening and an interior cavity adapted to receive an intravascular device therein;
a dilator tip movably disposed within the distal section of the elongated member, said dilator tip including a proximal section, a distal section, a distal end, and an inner lumen therethrough; and
a retraction wire operatively coupled to the dilator tip, said retraction wire configured to actuate the dilator tip between a first position disposed within the distal opening at least in part distally of the elongated member and a second position disposed within the interior cavity of the elongated member.

2. The retrieval device of claim 1, wherein the intermediate section of said elongated member defines a first lumen adapted to slidably receive the retrieval wire and a second lumen adapted to slidably receive a guidewire.

3. The retrieval device of claim 1, wherein the intermediate section of said elongated member includes a side guidewire port.

4. The retrieval device of claim 1, wherein the distal section of said elongated member is tapered along at least a portion of its length.

5. The retrieval device of claim 1, wherein the distal section of said elongated member is feathered along at least a portion of its length.

6. The retrieval device of claim 1, further comprising a guiding member coupled to the dilator tip, said guiding member having a proximal end, a distal end, and an inner lumen therethrough adapted to slidably receive a guidewire.

7. The retrieval device of claim 6, wherein the distal end of the guiding member terminates proximally of the distal end of the dilator tip.

8. The retrieval device of claim 6, wherein the guiding member includes an inner layer and an outer layer.

9. The retrieval device of claim 8, wherein the guiding member further includes at least one intermediate layer disposed between the inner and outer layers.

10. The retrieval device of claim 1, wherein the proximal section of said dilator tip is dimensioned to provide an interference fit with the distal opening of the elongated member.

11. The retrieval device of claim 1, wherein the distal section of the dilator tip is tapered.

12. The retrieval device of claim 1, further comprising means for engaging the retraction wire.

13. The retrieval device of claim 12, wherein said means for engaging the retraction wire includes a slide mechanism.

14. The retrieval device of claim 1, further comprising means for deflecting the dilator tip within the distal opening.

15. The retrieval device of claim 14, wherein said means for deflecting the dilator tip within the distal opening comprises an inflatable balloon cuff operatively coupled to the distal section of the elongated member.

16. The retrieval device of claim 1, wherein said means for deflecting the dilator tip within the distal opening comprises a tip deflection wire operatively coupled to the dilator tip.

17. The retrieval device of claim 1, wherein the elongated member includes at least one radiopaque marker band.

18. The retrieval device of claim 1, wherein the dilator tip comprises a radiopaque material.

19. The retrieval device of claim 1, wherein said intravascular device is an embolic protection filter.

20. A retrieval device, comprising:
an elongated member having a proximal section, an intermediate section, and a distal section, said distal section defining a distal opening and an interior cavity adapted to receive an intravascular device therein;
a dilator tip movably disposed within the distal section of the elongated member, said dilator tip including a proximal section, a distal section, a distal end, and an inner lumen therethrough;
a guiding member coupled to the dilator tip, said guiding member having a proximal end, a distal end, and an inner lumen therethrough adapted to slidably receive the guidewire; and
a retraction wire operatively coupled to the dilator tip, said retraction wire configured to actuate the dilator tip between a first position disposed within the distal opening at least in part distally of the elongated member and a second position disposed within the interior cavity of the elongated member.

21. The retrieval device of claim 20, wherein the intermediate section of said elongated member defines a first lumen adapted to slidably receive the retrieval wire and a second lumen adapted to slidably receive the guiding member and a guidewire.

22. The retrieval device of claim 20, wherein the intermediate section of said elongated member includes a side guidewire port.

23. The retrieval device of claim 20, wherein the distal section of said elongated member is tapered along at least a portion of its length.

24. The retrieval device of claim 20, wherein the distal section of said elongated member is feathered along at least a portion of its length.

25. The retrieval device of claim 20, wherein the distal end of the guiding member terminates proximally of the distal end of the dilator tip.

26. The retrieval device of claim 20, wherein the guiding member includes an inner layer and an outer layer.

27. The retrieval device of claim 26, wherein the guiding member further includes at least one intermediate layer disposed between the inner and outer layers.

28. The retrieval device of claim 20, wherein the proximal section of said dilator tip is dimensioned to provide an interference fit with the distal opening of the elongated member.

29. The retrieval device of claim 20, wherein the distal section of the dilator tip is tapered.

30. The retrieval device of claim 20, further comprising means for engaging the retraction wire.

31. The retrieval device of claim 30, wherein said means for engaging the retraction wire includes a slide mechanism.

32. The retrieval device of claim 20, further comprising means for deflecting the dilator tip within the distal opening.

33. The retrieval device of claim 32, wherein said means for deflecting the dilator tip within the distal opening comprises an inflatable balloon cuff operatively coupled to the distal section of the elongated member.

34. The retrieval device of claim 32, wherein said means for deflecting the dilator tip within the distal opening comprises a tip deflection wire operatively coupled to the dilator tip.

35. The retrieval device of claim 20, wherein the elongated member includes at least one radiopaque marker band.

36. The retrieval device of claim 20, wherein the dilator tip comprises a radiopaque material.

37. The retrieval device of claim 20, wherein said intravascular device is an embolic protection filter.

38. A retrieval device, comprising:
an elongated member having a proximal section, an intermediate section, and a distal section, said distal section defining a distal opening and an interior cavity adapted to receive an intravascular device therein;
a dilator tip movably disposed within the distal section of the elongated member, said dilator tip including a proximal section, a distal section, a distal end, and an inner lumen therethrough;
a retraction wire operatively coupled to the dilator tip, said retraction wire configured to actuate the dilator tip between a first position disposed within the distal opening at least in part distally of the elongated member and a second position disposed within the interior cavity of the elongated member; and
an inflatable balloon cuff operatively coupled to the distal section of the elongated member.

39. A retrieval device, comprising:
an elongated member having a proximal section, an intermediate section, and a distal section, said distal section defining a distal opening and an interior cavity adapted to receive an intravascular device therein;
a dilator tip movably disposed within the distal section of the elongated member, said dilator tip including a proximal section, a distal section, a distal end, and an inner lumen therethrough;
a retraction wire operatively coupled to the dilator tip, said retraction wire configured to actuate the dilator tip between a first position disposed within the distal opening at least in part distally of the elongated member and a second position disposed within the interior cavity of the elongated member; and
a tip deflection wire operatively coupled to the dilator tip.

40. A method of retrieving an intravascular device disposed along a guidewire within a patient's blood vessel, comprising the steps of:
providing a retrieval device equipped with retractable dilator tip and a retraction wire;
inserting the retrieval device into the body and advancing the retrieval device along the guidewire to a position proximal and adjacent to the intravascular device;
moving the dilator tip against a stop formed on the guidewire;
withdrawing the retraction wire proximally within the elongated member, causing the dilator tip and intravascular device to collapse at least in part within an interior cavity of the retrieval device; and
removing the retrieval device and collapsed intravascular device from the blood vessel.

41. The method of claim 40, wherein the retrieval device includes a slide mechanism for engaging the retraction wire, and wherein said step of withdrawing the retraction wire proximally is accomplished by moving the slide mechanism proximally.

42. The method of claim 40, wherein said intravascular device is an embolic protection filter.

43. A method of traversing an obstruction disposed along the inner wall of a patient's blood vessel, comprising the steps of:
providing a retrieval device equipped with retractable dilator tip, a retraction wire, and an inflatable balloon cuff;
inserting the retrieval device into the body and advancing the retrieval device along a guidewire to a position proximal the obstruction;
inflating the inflatable balloon cuff, causing the retractable dilator tip to center within the blood vessel; and
advancing the retrieval device beyond the obstruction.

44. The method of claim 43, further including the step of deflating the inflatable balloon cuff during the step of advancing the retrieval device beyond the obstruction.

45. The method of claim 43, further including the step of deflating the inflatable balloon cuff after the step of advancing the retrieval device beyond the obstruction.

46. The method of claim 43, further including the step of retrieving an intravascular device located distally of the obstruction.

47. A method of traversing an obstruction disposed along the wall of a patient's blood vessel, comprising the steps of:
providing a retrieval device equipped with retractable dilator tip, a retraction wire, and a tip deflection wire;
inserting the retrieval device into the body and advancing the retrieval device along a guidewire to a position proximal the obstruction;
engaging the tip deflection wire, causing the dilator tip to deflect away from the obstruction; and
advancing the retrieval device beyond the obstruction.

48. The method of claim 47, further including the step of retrieving an intravascular device located distally of the obstruction.
